(19) United States
(12) Patent Application Publication
(10) Pub. No.: US 2003/0223962 A1
(43) Pub. Date: Dec. 4, 2003

(54) Delivery of gene products to the lung parenchyma via gene transfer to the pleura

(75) Inventors: Ronald G. Crystal, New York, NY (US); Masahiro Mae, Tokyo (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Cornell Research Foundation, Inc., Ithaca, NY (US)

(21) Appl. No.: 10/161,356

(22) Filed: Jun. 3, 2002

(51) Int. Cl. A61K 48/00; A61K 38/19; C12N 15/861

(52) U.S. Cl. 424/93.2; 435/456; 424/85.1

(57) ABSTRACT

This invention pertains to a method of transferring a gene product to non-pleural tissue in a mammal, wherein a vector comprising an exogenous nucleic acid sequence which encodes a gene product is administered directly to the pleural cavity, the vector transfects pleural tissue cells, the exogenous nucleic acid sequence is expressed to produce the gene product, and the gene product contacts non-pleural tissue, thereby transferring the gene product to the non-pleural tissue.

### STATEMENT REGARDING FEDERALLY SPONSORED RESEARCH AND DEVELOPMENT

This invention was made in part with Government support under Grant Numbers 5R01HL57318-03 and 5R01HL61401-02 awarded by the National Heart, Lung, and Blood Institute. The Government may have certain rights in this invention.

### FIELD OF THE INVENTION

This invention pertains to the delivery of gene products and the treatment of disorders associated with non-pleural tissue in mammals.

### BACKGROUND OF THE INVENTION

Delivery of genes to the lung for the purposes of modifying the genetic repertoire of the lung for therapeutic purposes is very difficult for several reasons. The pulmonary epithelium is protected by the alveolar epithelial and bronchial fluid and the muco-ciliary escalator, which makes it difficult for gene transfer vectors administered by the airway route to reach the epithelium. Epithelial cells are deficient in many of the receptors used by viral vectors to bind to cells. The pulmonary epithelium is very sensitive, and toxicity is easily evoked by administration of viral or non-viral vectors to the respiratory epithelium. Moreover, the endothelial route of gene transfer to the lung is difficult because the blood is moving rapidly past the endothelium, and the endothelial cells are deficient in the receptors used by gene transfer vectors to bind to cells. For these reasons, the lung parenchyma presents a very challenging target for gene therapy, and there is no methodology available to use gene transfer to bathe the lung in an extracellular gene product for therapeutic purposes.

Since intravascular (e.g., intravenous) gene transfer is an ineffective strategy to deliver gene products to the lung parenchyma and intratracheal administration is associated with alveolar inflammation secondary to host defenses against viral vectors, a new strategy must be developed for the delivery of gene products to the lung for therapeutic purposes.

The invention provides such a means of delivery of genes to pleural tissue. These and other advantages of the invention, as well as additional inventive features, will be apparent from the description of the invention provided herein.

### BRIEF SUMMARY OF THE INVENTION

This invention pertains to a method of delivery of a gene product to non-pleural tissue and the treatment of disorders associated with non-pleural tissue in mammals. This method comprises directly administering a vector comprising an exogenous nucleic acid sequence that encodes a gene product to the pleural cavity of the lung. The vector transfects pleural tissue cells, and the exogenous nucleic acid sequence is expressed to produce the gene product. The gene product contacts non-pleural tissue to treat the disorder associated with the non-pleural tissue.

### BRIEF DESCRIPTION OF THE DRAWINGS

FIG. 1 is a graph of survival rate as a function of time (days after tumor injection) following the administration of Adsflt intrapleural (□), Adsflt nasal (●), Adsflt IV (∘), AdNull intrapleural (▪), and PBS intrapleural (Δ).

FIG. 2 is a graph of survival rate as a function of time (days after tumor injection) following no treatment (♦) or the administration of AdNull (▪) or AdPEDF (▴).

### DETAILED DESCRIPTION OF THE INVENTION

This invention pertains to a method of delivery of a gene product to non-pleural tissue and the treatment of disorders associated with non-pleural tissue in mammals. This method comprises directly administering a vector comprising an exogenous nucleic acid sequence that encodes a gene product to the pleural cavity of the lung. The vector transfects pleural tissue cells, and the exogenous nucleic acid sequence is expressed to produce the gene product. The gene product contacts non-pleural tissue to treat the disorder associated with the non-pleural tissue.

The invention uses lung pleural gene transfer to deliver gene products to the lung parenchyma (i.e., the cells and extracellular tissues of the lung other than the pleuraper se), as well as to deliver gene products to tissue outside of the lung. The advantages of using the pleura (pleural mesothelial cells and the cells underlying the visceral and parietal pleural mesothelium) are several. The pleural surface in the human (visceral and parietal combined) provide a large number of cells to be transduced or genetically modified. Unlike the pulmonary epithelium which is difficult to transduce and, very importantly, cannot be easily used as a target in humans because of the extreme sensitivity of the pulmonary epithelium to toxicity from gene transfer vectors, pleural mesothelial cells are easy to transduce or genetically modify, and toxicity (mostly due to host defense inflammatory responses to the gene transfer vectors) has little, if any, functional consequences to humans. Indeed, it is the general practice in medicine to induce inflammation in the pleura for therapy of recurrent pneumothorax or recurrent pleural effusions, and while this can be transiently painful, it has no significant functional consequences. In contrast, inflammation on the pulmonary epithelial surface can markedly affect lung function and can be fatal. Additionally, it is very easy for the clinician to gain access to the pleura using a needle and syringe, and thus it is easy to administer gene transfer vectors to this site. Other means of administration are also feasible using various procedures commonly used in clinical practice.

The pleural tissue is a serous membrane that covers the lung parenchyma, chest wall, and diaphragm with a single layer of flat cells. Pleural tissue cells are cells of the pleural mesothelium, cells adjacent to the pleural surface (e.g., the cells underlying the visceral and parietal pleural mesothelium), or a combination thereof. The pleural surface in the human (visceral and parietal combined) is approximately 4000 cm², containing approximately 1×10⁹ to 4×10⁹ mesothelial cells (Agostini et al., Respir. Physiol., 6, 330 (1969), and Sahn, Am. Rev. Respir. Dis., 138, 184-234 (1988)).

The cells of the pleura can be transfected or genetically modified with the use of a gene transfer vector. The vector administered to the pleura can be any suitable vector, including viral and non-viral vectors. Examples of suitable vectors include, for instance, plasmids, plasmid-liposome complexes, and viral vectors, e.g., parvoviral-based vectors (i.e., adeno-associated virus (AAV)-based vectors), retroviral vectors, herpes simplex virus (HSV)-based vectors, AAV-adenoviral chimeric vectors, and adenovirus-based vectors. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

Plasmids, genetically engineered circular double-stranded DNA molecules, can be designed to contain an expression cassette. Although plasmids were the first vector described for administration of therapeutic nucleic acids, the level of transfection efficiency is poor compared with other techniques. By complexing the plasmid with liposomes, the efficiency of gene transfer in general is improved. While the liposomes used for plasmid-mediated gene transfer strategies have various compositions, they are typically synthetic cationic lipids. Advantages of plasmid-liposome complexes include their ability to transfer large pieces of DNA encoding a therapeutic nucleic acid and their relatively low immunogenicity.

Plasmids are often used for short-term expression. However, a plasmid construct can be modified to obtain prolonged expression. It has recently been discovered that the inverted terminal repeats (ITR) of parvovirus, in particular adeno-associated virus (AAV), are responsible for the high-level persistent nucleic acid expression often associated with AAV (see, for example, U.S. Pat. No. 6,165,754). Accordingly, the gene transfer vector can be a plasmid comprising native parvovirus ITRs to obtain prolonged and substantial expression of a gene. While plasmids are suitable for use in the inventive method, preferably the transfer vector is a viral vector.

Retrovirus is an RNA virus capable of infecting a wide variety of host cells. Upon infection, the retroviral genome integrates into the genome of its host cell and is replicated along with host cell DNA, thereby constantly producing viral RNA and any nucleic acid sequence incorporated into the retroviral genome. When employing pathogenic retroviruses, e.g., human immunodeficiency virus (HIV) or human T-cell lymphotrophic viruses (HTLV), care must be taken in altering the viral genomic to eliminate toxicity. A retroviral vector can additionally be manipulated to render the virus replication-incompetent. As such, retroviral vectors are thought to be particularly useful for stable gene transfer in vivo. Lentiviral vectors, such as HIV-based vectors, are exemplary of retroviral vectors used for gene delivery. Unlike other retroviruses, HIV-based vectors are known to incorporate their passenger genes into non-dividing cells.

HSV-based viral vectors are suitable for use as gene transfer vectors to introduce nucleic acids into pleural cells. The mature HSV virion consists of an enveloped icosahedral capsid with a viral genome consisting of a linear double-stranded DNA molecule that is 152 kb. Most replication-deficient HSV vectors contain a deletion to remove one or more intermediate-early genes to prevent replication. Advantages of the herpes vector are its ability to enter a latent stage that can result in long-term DNA expression, and its large viral DNA genome that can accommodate exogenous DNA up to 25 kb. Of course, this ability is also a disadvantage in terms of short-term treatment regimens. For a description of HSV-based vectors appropriate for use in the present inventive methods, see, for example, U.S. Pat. Nos. 5,837,532, 5,846,782, 5,849,572, and 5,804,413 and International Patent Applications WO 91/02788, WO 96/04394, WO 98/15637, and WO 99/06583.

Preferred viral vectors include adenoviral vectors and adeno-associated vectors, which can readily transfer genes to the pleura. In contrast to adenoviral vectors, which transfer genes to the pleura transiently, adeno-associated viral vectors transfer genes to the pleura on a chronic basis (i.e., adeno-associated viral vectors can be used to provide gene products to the pleural surface, to the lung parenchyma, and to tissue outside of the lung on a chronic basis).

Adeno-associated virus (AAV) is a DNA virus, which is not known to cause human disease. AAV requires co-infection with a helper virus (i.e., an adenovirus or a herpes virus), or expression of helper genes, for efficient replication. AAV vectors used for administration of a therapeutic nucleic acid have approximately 96% of the parental genome deleted, such that only the terminal repeats (ITRs), which contain recognition signals for DNA replication and packaging, remain. This eliminates immunologic or toxic side effects due to expression of viral genes. In addition, delivering the AAV rep protein enables integration of the AAV vector comprising AAV ITRs into a specific region of genome, if desired. Host cells comprising an integrated AAV genome show no change in cell growth or morphology (see, for example, U.S. Pat. No. 4,797,368). Although efficient, the need for helper virus or helper genes can be an obstacle for widespread use of this vector.

Adenovirus (Ad) is a 36 kb double-stranded DNA virus that efficiently transfers DNA in vivo to a variety of different target cell types. Adenoviral vectors can be produced in high titers and can efficiently transfer DNA to replicating and non-replicating cells. The newly transferred genetic information remains epi-chromosomal, thus eliminating the risks of random insertional mutagenesis and permanent alteration of the genotype of the target cell. However, if desired, the integrative properties of AAV can be conferred to adenovirus by constructing an AAV-Ad chimeric vector. For example, the AAV ITRs and nucleic acid encoding the Rep protein incorporated into an adenoviral vector enable the adenoviral vector to integrate into a mammalian cell genome. In the context of the invention, the adenoviral vector can be derived from any serotype of adenovirus. Adenoviral stocks that can be employed as a source of adenovirus can be amplified from the adenoviral serotypes 1 through 51, which are currently available from the American Type Culture Collection (ATCC, Manassas, Va.), or from any other serotype of adenovirus available from any other source. Adenoviral vectors are described in, for example, U.S. Pat. Nos. 5,559,099, 5,712,136, 5,731,190, 5,837,511, 5,846,782, 5,851,806, 5,962,311, 5,965,541, 5,981,225, 5,994,106, 6,020,191, and 6,113,913, International Patent Applications WO 95/34671, WO 97/21826, and WO 00/00628, and Thomas Shenk, “Adenoviridae and their Replication,” and M. S. Horwitz, “Adenoviruses,” Chapters 67 and 68, respectively, in Virology, B. N. Fields et al., eds., 3d ed., Raven Press, Ltd., New York (1996).

When a viral vector is used (e.g., an adenoviral vector), it is preferred that the viral vector is deficient in at least one gene function required for viral replication, resulting in a “replication-deficient” viral vector. For example, when the viral vector is an adenoviral vector, it is preferable that the vector is deficient in at least one essential gene function of the E1 region (e.g., the E1a region and/or the E1b region), the E2 region, the E4 region, and/or any one or more of the L1-L5 regions of the adenoviral genome. Alternatively, or in addition, the recombinant adenovirus can be deficient in part or all of the E3 region (e.g., an Xba I deletion of the E3 region) and/or can have a mutation in the major late promoter (MLP), as discussed in International Patent Application WO 00/00628. Preferably, the adenoviral vector is “multiply deficient,” meaning that the adenoviral vector is deficient in one or more essential gene functions required for viral replication in each of two or more regions. For example, an E1-deficient or E1-, E3-deficient adenoviral vector can be further deficient in at least one essential gene of the E4 region and/or at least one essential gene of the E2 region (e.g., the E2A region). An adenoviral vector deleted of the entire E4 region can elicit a lower host immune response.

The construction of viral vectors is well understood in the art. Adeno-associated viral vectors can be constructed and/or purified using the methods set forth, for example, in U.S. Pat. No. 4,797,368 and Laughlin et al., Gene, 23, 65-73 (1983). Adenoviral vectors can be constructed and/or purified using the methods set forth, for example, in U.S. Pat. Nos. 5,851,806, 5,965,358, and 5,994,106 and International Patent Applications WO 95/34671, WO 98/56937, WO 99/15686, WO 99/54441, and WO 00/12765.

The vector (e.g., adenoviral vector) can be subject to any number of additional or alternative modifications. For example, the adenoviral vector may be a replication-deficient adenoviral vector that includes or produces (by expression) a modified adenoviral protein, non-adenoviral protein, or both, which increases the efficiency that the vector infects cells as compared to wild-type adenovirus, allows the vector to infect cells which are not normally infected by wild-type adenovirus, results in a reduced host immune response in a mammalian host as compared to wild-type adenovirus, or any combination thereof. Any suitable type of modification can be made to the vector, and many suitable modifications are known in the art. For example, the adenoviral vector coat protein can be modified, such as by altering the adenoviral fiber, penton, pIX, pIIIa, and/or hexon proteins, and/or by inserting a native or non-native ligand into one or more portions of such coat proteins. Manipulation of such coat proteins can broaden the range of cells infected by a viral vector (e.g., the vector can bind to and enter a broader range of eukaryotic cells than the corresponding wild-type virus), reduce the immune response to a viral vector, or enable targeting of a viral vector to a specific cell type. Examples of adenoviral vectors including such modifications to broaden the range of infected cells are described in International Patent Application WO 97/20051. Reduction of the immune response against an adenoviral vector can be obtained through the methods described in U.S. Pat. Nos. 6,093,699 and 6,211,160. Other adenoviral vector protein modifications that decrease the potential for immunological recognition by the host and resultant coat-protein directed neutralizing antibody production, are described in, for example, International Patent Applications WO 98/40509 and WO 00/34496. The manipulation of the viral coat such that the virus is “targeted” to a particular cell type (e.g., cells expressing unique receptors) is described in Miller et al., FASEB J., 9, 190-99 (1995), Douglas et al., Nat. Biotechnol., 14(11), 1574-78 (1996), Wickam, Gene Ther., 7(2), 110-14 (2000), U.S. Pat. Nos. 5,559,099, 5,731,190, 5,712,136, 5,770,442, 5,846,782, 5,962,311, 5,965,541, 5,985,655, 6,030,954, and 6,057,155 and International Patent Applications WO 96/07734, WO 96/26281, WO 97/20051, WO 98/07865, WO 98/07877, WO 98/40509, WO 98/54346, WO 00/15823, and WO 00/31285. In non-viral vector systems, the use of targeting through targeted proteins (e.g., an asialoorosomucoide protein conjugate which promotes liver targeting (such as is described in Wu et al., J. Biol. Chem., 263(29), 14621-24 (1988), or the targeted cationic lipid compositions of U.S. Pat. No. 6,120,799) is known.

The vector (e.g., adenoviral vector) also can include a trans-acting factor, cis-acting factor, or both, which preferably increases the persistence of transgene expression from the vector's genome. Any suitable trans-acting factor can be used, such as HSV ICPO, which prolongs transgene expression. Such modifications are particularly preferred in E4-deleted adenoviral vectors. The use of trans-acting factors is further described in International Patent Application WO 00/34496. Additionally or alternatively, the non-enveloped viral vector comprises a nucleic acid sequence encoding a cis-acting factor. For example, a matrix attachment region (MAR) sequence (e.g., an immunoglobulin heavy chain m (as discussed in, e.g., Jenuwein et al., Nature, 385(16), 269 (1997)), locus control region (LCR) sequences, or apolipoprotein B sequence (as discussed in, e.g., Kalos et al., Molec. Cell. Biol., 15(1), 198-207 (1995)) can be used to modify the persistence of expression from a transgene, such as a transgene inserted into an E4-deleted region of the adenoviral vector genome. LCR sequences are also believed to establish and/or maintain transcription of transgenes in a cis manner.

The exogenous nucleic acid sequence of the vector encodes a gene product that can be any suitable gene product. Preferably, the gene product is beneficial (e.g., prophylactically or therapeutically beneficial) to the non-pleural tissue (e.g., cell, tissue, organ, organ system, organism, or cell culture of which the non-pleural tissue is a part). If the gene product confers a prophylactic or therapeutic benefit to the non-pleural tissue, the gene product can exert its effect at the level of RNA or protein. For example, the gene product can encode a protein that can be employed in the treatment of an inherited disease (e.g., the cystic fibrosis transmembrane conductance regulator can be employed in the treatment of cystic fibrosis). Alternatively, the gene product can encode an antisense molecule, a ribozyme, a protein that affects splicing or 3′ processing (e.g., polyadenylation), or a protein that affects the level of expression of another gene within the cell (i.e., where gene expression is broadly considered to include all steps from initiation of transcription through production of a process protein), such as by mediating an altered rate of mRNA accumulation or transport or an alteration in post-transcriptional regulation. Since treatment of non-pleural tissue is predicated upon the transfer of the gene product from the pleural cavity to the non-pleural tissue, it is preferred that the exogenous nucleic acid encodes a secreted peptide (e.g., α₁-antitrypsin).

The exogenous nucleic acid sequence desirably encodes a gene product that is anti-angiogenic. Suitable anti-angiogenic factors include pigment epithelium-derived factor (PEDF), angiostatin, vasculostatin, endostatin, platelet factor 4, heparinase, interferons (e.g., INFa), and the like. One of ordinary skill in the art will appreciate that any anti-angiogenic factor can be modified or truncated and retain anti-angiogenic activity. As such, the exogenous nucleic acid sequence can encode a gene product that is an active fragment of an anti-angiogenic factor (i.e., a fragment having biological activity sufficient to inhibit angiogenesis).

A preferred anti-angiogenic factor is PEDF, also named early population doubling factor-1 (EPC-1). PEDF is a secreted protein having homology to a family of serine protease inhibitors named serpins. PEDF is further characterized in U.S. Pat. No. 5,840,686 and International Patent Applications WO 93/24529 and WO 99/04806. Anti-angiogenic derivatives of PEDF include SLED proteins, described in International Patent Application WO 99/04806. It has also been postulated that PEDF is involved with cell senescence (Pignolo et al., J. Biol. Chem., 268 (12), 8949-8957 (1998)).

Another preferred exogenous nucleic acid sequence is that which encodes the flt-1 receptor for vascular endothelial growth factor (VEGF). More preferably, the exogenous nucleic acid sequence encodes sflt (an extracellular form of the flt-1 receptor for VEGF which is soluble and secreted). Various forms of the flt-1 receptor are anti-angiogenic (see, e.g., Takayama et al., Canc. Res., 60, 2169-2177 (2000); Roeckl et al., Exp. Cell Res., 241, 161-170 (1998); Hiratsuka et al., Proc. Natl. Acad. Sci. USA, 95, 9349-9354 (1998)).

Another preferred exogenous nucleic acid sequence is that which encodes tumor necrosis factor (TNF), especially TNF-α, which is well known for its anti-tumor effects and ability to act synergistically with radiation therapy. Substances that enhance the local effect of TNF can be used to reduce the level of TNF required to produce a prophylactic or therapeutic effect in a host. Such substances include TNF antagonists, for example, soluble TNF receptors, anti-TNF antibodies, and TNF agonists. Other suitable antagonists, agonists, and other substances that alter the effect of TNF are available and generally known in the art.

The transfer of the vector to the pleural cavity can be done by any suitable technique, many of which are commonly used in clinical practice. A preferred method of intrapleural administration is by the use of injection with needle and syringe.

The inventive method of transferring a gene product to non-pleural tissue in a mammal can be utilized for several purposes. For example, the inventive method can be used as a research tool to study vector development, to screen libraries, and to study mammalian systems, such as mammalian immune response. A preferred utility for the inventive method is the treatment of non-pleural disorders.

The non-pleural disorder to be treated by the inventive method can be any suitable disorder. Suitable disorders include, but are not limited to, cancer, cystic fibrosis, asthma, chronic bronchitis, and interstitial lung diseases (e.g., bronchiolitis, alveolitis, vasculitis). Preferably, the disorder is cancer (e.g., lung cancer, metastases of the lung from cancers of origin other than lung). Desirably, the mammal has a tumor, and the contacting of the non-pleural tissue with the gene product results in a reduction in size of the tumor.

The non-pleural tissue can be any tissue of the mammal. Preferably, the non-pleural tissue is lung parenchyma. Additional non-pleural tissues include, but are not limited to, liver, spleen, muscle tissue, and a combination thereof.

The inventive method can be used to transfer a gene product to non-pleural tissue in any mammal. Mammals include those routinely used in research, including, but not limited to, mice, rats, rabbits, canines, swine, and primates. Preferably, the mammal is a human.

The following examples further illustrate the invention but, of course, should not be construed as in any way limiting its scope.

### EXAMPLE 1

This example illustrates the ability of intrapleural gene transfer to modify lung parenchymal processes.

CT26.CL25 tumor cells were derived from CT26 colon adenocarcinoma cells that had been modified to express the E. coli β-galactosidase (β-gal) gene. 3×10⁵ CT26.CL25 tumor cells were injected into BALB/c mice via the jugular vein to generate tumor metastases in the lung parenchyma of the mice. Twenty-four hours following tumor cell injection, each of the mice was administered 5×10⁸ particle forming units (pfu) of an adenoviral vector (Adsflt or AdNull) or phosphate buffered saline (PBS). The adenoviral vector was E1-, E3-deficient and contained an expression cassette in the E1 region including the cytomegalovirus early/intermediate promoter/enhancer (CMV promoter). Adsflt contained an exogenous nucleic acid sequence encoding a soluble, secreted, extracellular portion of the flt-1 receptor for vascular endothelial growth factor (VEGF) within the expression cassette. AdNull did not contain the transgene but was otherwise identical to Adsflt. Adsflt, AdNull, or PBS was administered to the mice by an intrapleural, intranasal, or intravenous route. Twelve days following tumor injection, the lungs of the mice were harvested, weighed, and measured for β-gal activity. Both total weight and total β-gal activity were used as parallel measures of total tumor burden of the CT26.CL25 colon carcinoma cells expressing β-gal. The β-gal reporter gene in the CT26.CL25 colon carcinoma cells was assessed in lung lysates in a luminometer (e.g., Promega Corp., Madison, Wis.). β-gal activity in the lung was expressed as total activity per lung and relative to lung protein content determined using the BCA protein assay (e.g., Bio-Rad Laboratories, Hercules, Calif.). The resulting data, represented as the mean for 5 to 6 mice per group, are set forth in Tables 1 and 2.

TABLE 1
Lung wet weight (mg) following injection with CT26.CL25 tumor cells
and administration of Adsflt or controls by different routes.

| | Right Lung | Left Lung |
|---|---|---|
| PBS (intrapleural) | 400 | 150 |
| AdNull (intrapleural) | 360 | 180 |
| Adsflt (intrapleural) | 150 | 80 |
| Adsflt (intranasal) | 250 | 100 |
| Adsflt (intravenous) | 170 | 70 |
| Naïve (no tumor cell | 90 | 60 |
| injection or therapy) | | |

TABLE 2
Lung β-galactosidase activity (×10⁷ RLU/mg protein) following
injection with CT26.CL25 tumor cells (expressing β-gal) and
administration of Adsflt or controls by different routes.

| | Right Lung | Left Lung |
|---|---|---|
| PBS (intrapleural) | 13 | 8 |
| AdNull (intrapleural) | 16 | 13 |
| Adsflt (intrapleural) | 2 | 2 |
| Adsflt (intranasal) | 4 | 2 |
| Adsflt (intravenous) | 4 | 2 |
| Naïve (no tumor cell | 0 | 0 |
| injection or therapy) | | |

The data in Tables 1 and 2 illustrate that intrapleural administration of Adsflt results in a marked suppression of tumor growth in the parenchyma of both lungs when compared to intrapleural administration of PBS or AdNull controls or the intranasal or intravenous administration of Adsflt. Lung weights were lower in mice that had been intrapleurally administered Adsflt as compared to intrapleural administration of PBS or AdNull or the intranasal or intravenous administration of Adsflt. Additionally, the lung β-gal activity (representing CT26.CL25 tumor cell activity) was lower in mice which had been intrapleurally administered Adsflt instead of PBS or AdNull or which had been intranasally or intravenously administered Adsflt.

The resulting data of this example demonstrate the benefits of treatment of a non-pleural disorder (e.g., lung metastases) by intrapleural administration of a gene transfer vector in accordance with the inventive method.

### EXAMPLE 2

This example illustrates increased survival of mammals with lung metastases treated in accordance with the inventive method of administering a therapeutic gene transfer vector to the lung pleural cavity.

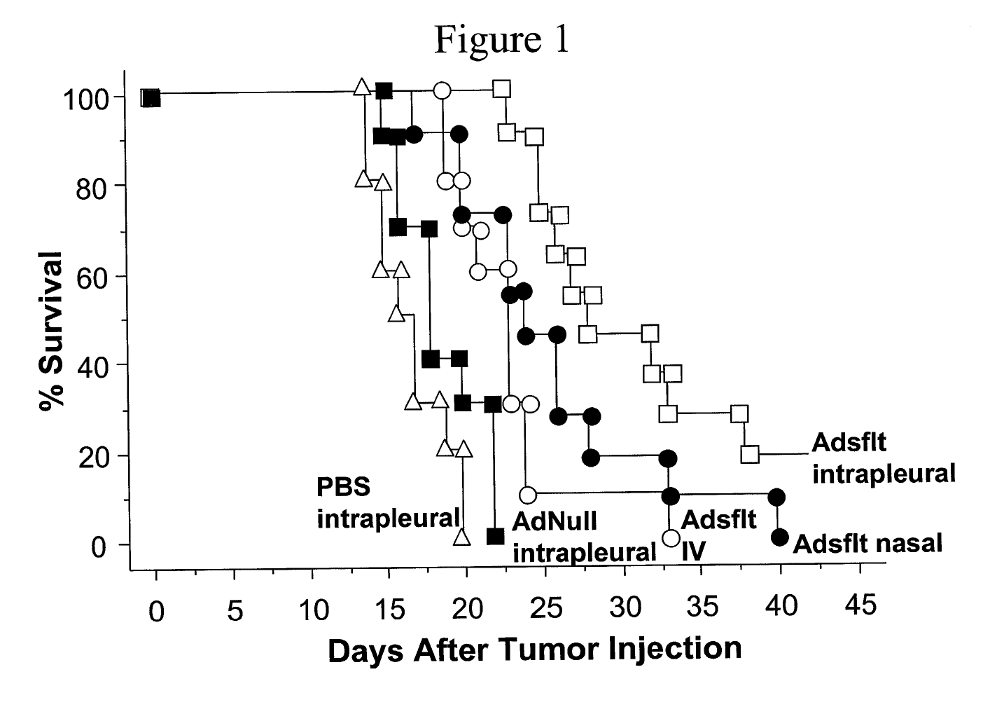

Mice were injected with 3×10⁵ CT26.CL25 tumor cells. One day later, the mice were administed of 5×10⁸ pfu Adsflt, 5×10⁸ pfu AdNull, or PBS by an intrapleural, intranasal, or intravenous route as described in Example 1. The survival of animals was recorded as the percentage of live animals remaining in each group (10 to 11 mice per group) at different times after the tumor cell injection. Those survival percentages are plotted in the graph of FIG. 1.

The data in FIG. 1 demonstrate that the animals administered Adsflt intrapleurally had the best survival percentage. At the last time point studied, 38 days after tumor injection, about 20% of the mice survived. In distinct contrast, no mice administered PBS intrapleurally, AdNull intrapleurally, Adsflt intravenously, or Adsflt nasally survived beyond 38 days post-tumor injection. In particular, following intrapleural administration with PBS, there were no mice surviving at day 20. Intrapleural AdNull administration resulted in no mice surviving at day 22. When Adsflt was administered intravenously or intranasally, there were no mice surviving at days 33 and 40, respectively.

The results of this example demonstrate the treatment of a non-pleural disorder (e.g., lung metastases) by intrapleural administration of a vector comprising an exogenous nucleic acid sequence that encodes a gene product.

### EXAMPLE 3

This example illustrates the effects of intrapleural administration of a therapeutic gene transfer vector on the progression of preexisting distant (non-lung) tumors.

CT26.CL25 tumor cells (3×10⁵) were injected subcutaneously in the right flank of BALB/c mice. On day 5, tumor-bearing mice were treated by intravenous, intranasal, intrapleural, or intratumoral injection of 5×10⁸ pfu of the therapeutic gene transfer vector Adsflt (described in Example 1). The size of the subcutaneous tumors was measured every other day and recorded as the average tumor area (mm²) by measuring the largest perpendicular diameters. Control animals were tumor-bearing mice without any treatment (naïve animals). The resulting data, represented as the mean for 5 to 6 mice per group, are set forth in Table 3.

TABLE 3
Tumor size (mm²) following administration
of Adsflt by different routes.

| Days | | | | | |
|---|---|---|---|---|---|
| Following | | | | | |
| Tumor | | | | | In- |
| Implantation | Naive | Intravenous | Intranasal | Intrapleural | tratumoral |
| 0 | 0 | 0 | 0 | 0 | 0 |
| 5 | 23 | 23 | 23 | 23 | 23 |
| 7 | 47 | 38 | 32 | 32 | 30 |
| 9 | 56 | 49 | 49 | 40 | 36 |
| 11 | 79 | 61 | 61 | 48 | 40 |
| 13 | 88 | 78 | 70 | 60 | 50 |
| 15 | 111 | 92 | 92 | 74 | 56 |
| 17 | 136 | 120 | 117 | 96 | 59 |
| 19 | 160 | 157 | 143 | 130 | 69 |

The data set forth in Table 3 indicate that intrapleural and intratumoral administration of Adsflt have the greatest effect on the reduction of the size of the right flank tumors. Additionally, the data demonstrate that the suppression of lung parenchymal metastases by intrapleural Adsflt is not mediated by immunologic processes that merely function at the site of gene transfer, since intrapleural Adsflt administration suppresses the growth of the distant (non-lung) tumors.

The data resulting for this example demonstrate the treatment of a non-pleural disorder (e.g., non-lung (specifically right flank) tumors) by intrapleural administration of a vector comprising an exogenous nucleic acid sequence that encodes a gene product.

### EXAMPLE 4

This example provides a quantitative analysis of tissue distribution of luciferase activity in mice following the administration by different routes of an adenoviral vector coding for an intracellular protein (luciferase; AdLuc).

Luciferase activity in homogenates of different individual organs (right lung, left lung, diaphragm, liver, spleen, and skeletal muscle) was assessed 3 days after administration of 1×10⁸ pfu of AdLuc by intrapleural, intratracheal, and intravenous routes to BALB/c mice. Luciferase activities were determined as relative light units (RLU) quantified in a luminometer (e.g., Promega Corp., Madison, Wis.) and standardized by total protein concentration using the BCA assay. The resulting data are set forth in Table 4.

TABLE 4
Relative light units per milligram of protein detected in selected organs
following administration of AdLuc by different routes.

| | Intrapleural | Intratracheal | Intravenous |
|---|---|---|---|
| Right Lung | 1 × 10⁸ | 2 × 10⁷ | 1 × 10³ |
| Left Lung | 1 × 10⁸ | 6 × 10⁷ | 1 × 10³ |
| Right Diaphragm | 2 × 10⁷ | 3 × 10³ | 6 × 10³ |
| Liver | 2 × 10⁶ | 8 × 10³ | 6 × 10⁶ |
| Spleen | 1 × 10⁴ | b.d. | 5 × 10⁴ |
| Muscle (right | 4 × 10⁵ | b.d. | 2 × 10³ |
| quadriceps femoris) | | | |

b.d. = below the detectable level (1 × 10³ relative light units/mg protein is the limit of detection of the assay)

The data set forth in Table 4 demonstrate that intrapleural administration of AdLuc showed higher levels of luciferase in both the right and left lung, the diaphragm, and skeletal muscle, as compared to the intratracheal and intravenous routes. Unilateral intrapleural administration was found sufficient to transfer genes bilaterally to the pleura.

The results of this example demonstrate that the administration of a gene transfer vector into the pleural cavity mediates the production of the gene product in mesothelial cells, and the extracellular product(s) produced by these genetically modified cells reach the lung parenchyma as well as other organs.

### EXAMPLE 5

This example provides a quantitative analysis of tissue distribution of luciferase activity in mice following the administration by the intrapleural route of an adeno-associated vector encoding an intracellular protein (luciferase; AAV.Luc).

Luciferase activity in homogenates of different individual organs (right lung, left lung, diaphragm, liver, spleen, and skeletal muscle) was assessed at 3, 14, 30, and 60 days after administration of 1×10¹⁰ particle units of AAV.Luc by the intrapleural route to BALB/c mice. The resulting data are set forth in Table 5.

TABLE 5
Relative light units per milligram of protein detected in selected organs
following administration of AAV.Luc.

| | Day 3 | Day 14 | Day 30 | Day 60 |
|---|---|---|---|---|
| Right Lung | b.d. | 3 × 10⁵ | 6 × 10⁵ | 4 × 10⁵ |
| Left Lung | b.d. | 9 × 10⁴ | 4 × 10⁵ | 4 × 10⁵ |
| Right Diaphragm | 4 × 10⁵ | 2 × 10⁷ | 1 × 10⁷ | 2 × 10⁷ |
| Liver | 6 × 10⁴ | 6 × 10⁴ | 4 × 10⁴ | 3 × 10⁴ |
| Spleen | b.d. | b.d. | b.d. | b.d. |
| Muscle (right | b.d. | b.d. | b.d. | b.d. |
| quadriceps | | | | |
| femoris) | | | | |

b.d. = below the detectable level (7 × 10³ relative light units/mg protein is the limit of detection of the assay)

The data set forth in Table 5 demonstrate that intrapleural administration of AAV vectors can be used for chronic gene expression and protein delivery to non-pleural tissues. At the last time point studied (60 days following AAV.Luc administration), the amount of luciferase detected in the right and left lungs, as well as other organs, remains relatively constant when compared to day 14, indicating chronic gene expression and gene product production. Moreover, the data also indicate that intrapleural administration of AAV.Luc results in high levels of luciferase in both the right and left lung. Unilateral intrapleural administration was found sufficient to transfer genes bilaterally to the pleura.

The results of this example demonstrate that the administration of a gene transfer vector into the pleural cavity mediates production of the gene product in mesothelial cells, and the extracellular product(s) produced by these genetically modified cells reach the lung parenchyma. Moreover, gene transfer vectors such as adeno-associated viral vectors are demonstrated to be capable of transferring genes on a chronic basis, so that gene transfer vectors can be used to provide gene products to the pleural surface, to the lung parenchyma, and to the area outside of the lung on a chronic basis.

### EXAMPLE 6

This example illustrates increased survival of mammals with lung metastases treated in accordance with the inventive method of administering a gene transfer vector to the lung pleural cavity to treat a disorder.

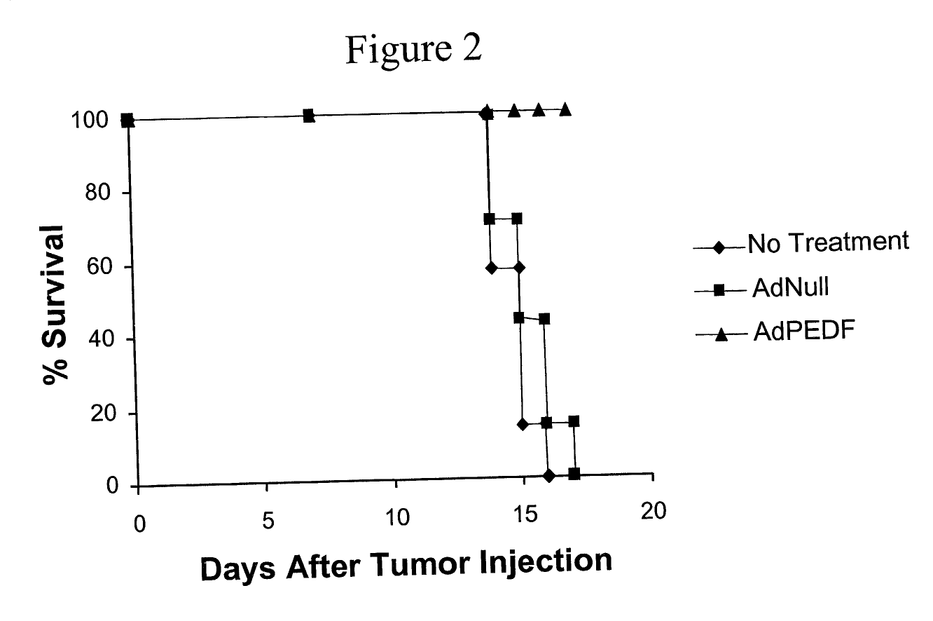

Mice were injected intraveneously with 3×10⁵ CT26.CL25 tumor cells. One day later, the mice were intrapleurally administered 1×10⁸ pfu of the adenoviral vector AdPEDF or AdNull, or received no treatment. The adenovectors AdPEDF and AdNull were similar to the adenovectors Adsflt and AdNull described in Example 1, except that in AdPEDF the exogenous nucleic acid sequence encoded pigment epithelium-derived factor (PEDF), whereas in Adsflt the exogenous nucleic acid sequence encoded a soluble, secreted, extracellular portion of the flt-1 receptor (sflt). The survival of animals was recorded as the percentage of live animals remaining in each group at different times after the tumor cell injection. Those survival percentages are plotted in the graph of FIG. 2.

The data in FIG. 2 demonstrate that the animals administered AdPEDF intrapleurally exhibited the best survival percentage. At the last time point studied, 17 days after tumor injection, 100% of the mice that were intrapleurally administered AdPEDF survived. In distinct contrast, no mice that were administered AdNull intrapleurally or received no treatment survived beyond 17 days post-tumor injection. In particular, there were no mice surviving at day 16 when no treatment was administered post-tumor injection. Intrapleural AdNull administration resulted in no mice surviving at day 17.

The results of this example demonstrate the effective treatment of a non-pleural disorder (e.g., lung metastases) by intrapleural administration of a vector comprising an exogenous nucleic acid sequence that encodes a gene product (e.g., PEDF).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of transferring a gene product to non-pleural tissue in a mammal, wherein a vector comprising an exogenous nucleic acid sequence which encodes a gene product is administered directly to the pleural cavity, the vector transfects pleural tissue cells, the exogenous nucleic acid sequence is expressed to produce the gene product, and the gene product contacts non-pleural tissue, thereby transferring the gene product to the non-pleural tissue.

2. The method of claim 1, wherein the pleural tissue cells are cells of the pleural mesothelium, cells adjacent to the pleural surface, or a combination thereof.

3. The method of claim 1, wherein the exogenous nucleic acid sequence encodes a secreted peptide.

4. The method of claim 1, wherein the exogenous nucleic acid sequence encodes flt-1.

5. The method of claim 3, wherein the exogenous nucleic acid sequence encodes sflt.

6. The method of claim 3, wherein the exogenous nucleic acid sequence encodes PEDF.

7. The method of claim 3, wherein the exogenous nucleic acid sequence encodes TNF.

8. The method of claim 7, wherein the exogenous nucleic acid sequence encodes TNF-α.

9. The method of claim 1, wherein the vector is a viral vector.

10. The method of claim 9, wherein the viral vector an adenoviral vector.

11. The method of claim 10, wherein the adenoviral vector is replication-deficient.

11. The method of claim 9, wherein the viral vector is an adeno-associated viral vector.

12. The method of claim 1, wherein the mammal is a human.

13. The method of claim 1, wherein the non-pleural tissue is lung parenchyma.

14. The method of claim 1, wherein the non-pleural tissue has a disorder, and the contacting of the non-pleural cells with the gene product leads to treatment of the disorder.

15. The method of claim 14, wherein the non-pleural disorder is cancer.

16. The method of claim 15, wherein the mammal has a tumor, and the contacting of non-pleural tissue with the gene product results in a reduction in size of the tumor.
